(19) United States
(12) Patent Application Publication
(10) Pub. No.: US 2026/0266774 A1
(43) Pub. Date: Sep. 10, 2026

(54) COMPOSITION AND SURROGATE MATRIX FOR PRESERVING VITAMIN D AND VITAMIN K AND APPLICATION METHOD THEREOF

(71) Applicant: Beijing Jishuitan Hospital, Capital Medical University, Beijing (CN)

(72) Inventors: Chunyan Li, Beijing (CN); Jun Wu, Beijing (CN); Hailing Chen, Beijing (CN); Chengeng Liu, Beijing (CN); Jie Yang, Beijing (CN); Renxian Wang, Beijing (CN); Ruming Liu, Botou (CN)

(21) Appl. No.: 19/437,200

(22) Filed: Dec. 30, 2025

(30) Foreign Application Priority Data
Mar. 5, 2025 (CN) 202510254224.3

(51) Int. Cl. G01N 30/06; C12N 9/18; G01N 30/02; G01N 30/04; G01N 30/88

(52) U.S. Cl. CPC G01N 30/06; C12N 9/18; C12Y 301/01034; G01N 2030/027; G01N 2030/042; G01N 2030/884

(57) ABSTRACT

A composition and a surrogate matrix for preserving vitamin D and vitamin K, and an application method thereof are provided, relating to the technical field of detection reagents. The composition includes sodium metabisulfite with a concentration in a range of 0.5 mol/L to 3 mol/L, sodium bisulfite with a concentration in a range of 0.5 mol/L to 3 mol/L, and lipoprotein lipase with a concentration in a range of 0.5 weight percent (wt %) to 3 wt %. The composition relieves the technical problems that the vitamin D and the vitamin K are unstable and easy to fail.

### CROSS-REFERENCE TO RELATED APPLICATION

This application claims priority to Chinese Patent Application No. 202510254224.3, filed on Mar. 5, 2025, which is herein incorporated by reference in its entirety.

### TECHNICAL FIELD

The disclosure relates to the technical field of detection reagents, and more particularly to a composition and a surrogate matrix for preserving vitamin D and vitamin K and an application method thereof.

### BACKGROUND

The following statements are provided only for background information related to the disclosure and do not necessarily constitute the related art.

Vitamins are important compounds involved in regulating human metabolic processes and physiological functions. Vitamin D promotes absorption of calcium and phosphorus, and lack of the vitamin D can lead to deformity of growing bone cells, inducing rickets. Vitamin K is involved in coagulation, anticoagulation, regulation of bone metabolism, and cell growth and proliferation, as well as reducing a probability of occurrence of physiological and pathological reactions such as oxidative stress and inflammation.

Issues of stability of the vitamin D and the vitamin K have always been critical factors affecting accurate detection. The vitamin D and vitamin K are prone to reduced activity and degradation due to factors such as light exposure, temperature, humidity, and oxidation, posing challenges for clinical testing. Inherent properties influencing the stability of the vitamin D and the vitamin K include: (1) oxidation resistance: the vitamin D is sensitive to oxidants, while the vitamin K is not sensitive to the oxidants, and when exposed to the oxidants, a loss of the vitamin K is more than 60% after one week of storage at room temperature; (2) reduction resistance: the vitamin K is sensitive to reducing agents, while the vitamin D is not sensitive to the reducing agents, and when exposed to the reducing agents, a loss of the vitamin D is more than 30% after one week of storage at the room temperature; and (3) acid alkaline environment: neutral and weakly alkaline environments are beneficial for maintaining the stability of the vitamin D, and the neutral environment is beneficial for maintaining the stability of the vitamin K.

Environmental factors affecting the stability of the vitamin D and the vitamin K include: (1) trace elements, (2) enzymes, and (3) adsorption. (1) The trace elements: the trace elements in serum have a great influence on the vitamin D and the vitamin K, especially certain trace element sulfates with high crystallization water, such as ferrous sulfate heptahydrate and zinc sulfate heptahydrate. Some trace element additives are both oxidants and reducing agents, as well as promoters of certain oxidation processes (such as copper). In presence of the trace elements, some unstable vitamins are prone to failure, and the higher the concentrations of the trace elements and the longer the time, the more the loss of these vitamins. For example, it has been measured that the serum with extremely low trace element content only loses 17% of the vitamin K after being stored at room temperature for one week, while when the serum contains trace elements, the loss of the vitamin K can reach over 70%. (2) The enzymes: various enzymes in the serum also have a certain impact on the stability of the vitamins. The vitamins serve as coenzymes for enzymes to perform physiological functions. When an environmental temperature and a value of potential of hydrogen (pH) are suitable, the enzymes will consume the vitamins. (3) Adsorption: the vitamin K exhibits strong adsorption properties for carriers made of ordinary material and endogenous lipids. It has been reported that a recovery rate of the vitamin K after one week of storage in ordinary Eppendorf Tubes® (EP) is only 30%.

Accurate detection results are crucial for diagnosing vitamin D deficiency (e.g., rickets, osteoporosis) and vitamin K deficiency (e.g., coagulation disorders). Instability of the vitamin D and the vitamin K during a detection process can lead to deviation in detection results, causing false positives and false negatives, which affect accurate assessment of vitamin levels in patients. Therefore, how to improve the stability of the vitamin D and the vitamin K is an issue that needs to be addressed.

In view of this, the disclosure is proposed.

### SUMMARY

The disclosure aims to provide a composition for preserving vitamin D and vitamin K, to thereby relieve technical problems that the vitamin D and the vitamin K are unstable and easy to fail.

To address the aforementioned technical problems, the disclosure uses the following technical solutions.

In a first aspect, the composition for preserving the vitamin D and the vitamin K is provided by the disclosure, including sodium metabisulfite with a concentration in a range of 0.5 moles per liter (mol/L) to 3 mol/L, sodium bisulfite with a concentration in a range of 0.5 mol/L to 3 mol/L, and lipoprotein lipase with a concentration in a range of 0.5 weight percent (wt %) to 3 wt %.

In a second aspect, the disclosure provides an application method of the composition provided in the first aspect, including preserving the vitamin D and the vitamin K using the composition.

In a third aspect, the disclosure provides a preservation method for the vitamin D and the vitamin K. The preservation method includes adding the composition provided in the first aspect to a system preserving the vitamin D and the vitamin K.

In a fourth aspect, the disclosure provides a surrogate matrix for the vitamin D and the vitamin K. The surrogate matrix includes the composition provided in the first aspect and a diluent.

In a fifth aspect, the disclosure provides a preparation method for the surrogate matrix for the vitamin D and the vitamin K. The preparation method includes mixing the composition with the diluent.

In a sixth aspect, the disclosure provides a reagent containing the vitamin D and the vitamin K. The reagent includes predetermined amounts of the vitamin D and the vitamin K and the surrogate matrix for the vitamin D and the vitamin K provided in the fourth aspect.

In a seventh aspect, the disclosure provides a detection kit for the vitamin D and the vitamin K. The detection kit includes at least one of the composition provided in the first aspect, the surrogate matrix provided in the fourth aspect, and the reagent provided in the sixth aspect.

In an eighth aspect, the disclosure provides a detection method for the vitamin D and the vitamin K. The detection method includes using the composition provided in the first aspect during a detection process to reduce loss of the vitamin D and the vitamin K in a sample.

Compared with the related art, the disclosure has the following beneficial effects.

The composition for preserving the vitamin D and the vitamin K provided by the disclosure includes the sodium metabisulfite, the sodium bisulfite, and the lipoprotein lipase. Through a synergistic effect of these three components, the composition can alleviate degradation of the vitamin D and the vitamin K and prevent the vitamin D and the vitamin K from binding with proteins and/or fats in environment. Applying the composition to detection of the vitamin D and the vitamin K can prevent the vitamin D and the vitamin K in the sample to be tested and the surrogate matrix from degrading and binding with the proteins and/or fats, thereby improving accuracy of detection results. The composition provided by the disclosure is of great significance for maintaining stability of the vitamin D and the vitamin K during the detection process, improving the accuracy of the detection results, ensuring reliability of evaluation of therapeutic effects, improving laboratory detection quality, and promoting the clinical research.

### BRIEF DESCRIPTION OF DRAWINGS

In order to explain embodiments of the disclosure or technical solutions in the art more clearly, attached drawings needed in description of the embodiments or the technical solutions in the art will be briefly introduced below. Apparently, the attached drawings introduced below are part of the embodiments of the disclosure, and other drawings can be obtained according to these attached drawings without creative work for those skilled in the art.

FIG. 1 illustrates a chromatogram of vitamin D2 in experiment group 3 of embodiment 3.

FIG. 2 illustrates a chromatogram of vitamin D3 in the experiment group 3 of the embodiment 3.

FIG. 3 illustrates a chromatogram of vitamin K (menaquinone-4, MK-4) in the experiment group 3 of the embodiment 3.

FIG. 4 illustrates a chromatogram of vitamin K (menaquinone-4, MK-7) in the experiment group 3 of the embodiment 3.

FIG. 5 illustrates a chromatogram of the vitamin D2 in experiment group 7 of the embodiment 3.

FIG. 6 illustrates a chromatogram of the vitamin D3 in the experiment group 7 of the embodiment 3.

FIG. 7 illustrates a chromatogram of the vitamin K (MK-4) in the experiment group 7 of the embodiment 3.

FIG. 8 illustrates a chromatogram of the vitamin K (MK-7) in the experiment group 7 of the embodiment 3.

FIG. 9 illustrates a chromatogram of the vitamin D2 in experiment group 8 of the embodiment 3.

FIG. 10 illustrates a chromatogram of the vitamin D3 in the experiment group 8 of the embodiment 3.

FIG. 11 illustrates a chromatogram of the vitamin K (MK-4) in the experiment group 8 of the embodiment 3.

FIG. 12 illustrates a chromatogram of the vitamin K (MK-7) in the experiment group 8 of the embodiment 3.

FIG. 13 illustrates a chromatogram of the vitamin D2 in experiment group 12 of the embodiment 3.

FIG. 14 illustrates a chromatogram of the vitamin D3 in the experiment group 12 of the embodiment 3.

FIG. 15 illustrates a chromatogram of the vitamin K (MK-4) in the experiment group 12 of the embodiment 3.

FIG. 16 illustrates a chromatogram of the vitamin K (MK-7) in the experiment group 12 of the embodiment 3.

FIG. 17 illustrates a chromatogram of the vitamin D2 in experiment group 16 of the embodiment 3.

FIG. 18 illustrates a chromatogram of the vitamin D3 in the experiment group 16 of the embodiment 3.

FIG. 19 illustrates a chromatogram of the vitamin K (MK-4) in the experiment group 16 of the embodiment 3.

FIG. 20 illustrates a chromatogram of the vitamin K (MK-7) in the experiment group 16 of the embodiment 3.

### DETAILED DESCRIPTION OF EMBODIMENTS

Technical solutions of the disclosure will be described clearly and completely with reference to embodiments. Apparently, described embodiments are only part of the embodiments of the disclosure, not all of them. Based on the embodiments of the disclosure, all other embodiments obtained by those skilled in the art without creative labor shall fall within a scope of protection of the disclosure.

In a first aspect, the disclosure provides a composition for preserving vitamin D and vitamin K. The composition includes sodium metabisulfite, sodium bisulfite, and lipoprotein lipase. The sodium metabisulfite and the sodium bisulfite as reducing agents can alleviate degradation of the vitamin D and the vitamin K, and the lipoprotein lipase can prevent the vitamin D and the vitamin K from binding with proteins and/or fats in environment. In the composition, a concentration of the sodium metabisulfite is in a range of 0.5 mol/L to 3 mol/L, such as but not limited to 0.5 mol/L, 1 mol/L, 1.5 mol/L, 2 mol/L, 2.5 mol/L, or 3 mol/L; a concentration of the sodium bisulfite is in a range of 0.5 mol/L to 3 mol/L, such as but not limited to 0.5 mol/L, 1 mol/L, 1.5 mol/L, 2 mol/L, 2.5 mol/L, or 3 mol/L; and a concentration of the lipoprotein lipase is in a range of 0.5 wt % to 3 wt %, such as but not limited to 0.5 wt %, 1 wt %, 1.5 wt %, 2 wt %, 2.5 wt %, or 3 wt %.

In an embodiment, the composition includes the sodium metabisulfite with the concentration in a range of 0.5 mol/L to 2 mol/L, the sodium bisulfite with the concentration in a range of 0.5 mol/L to 2 mol/L, and the lipoprotein lipase with the concentration in a range of 0.5 wt % to 2 wt %.

In an embodiment, the composition includes the sodium metabisulfite with the concentration of 1 mol/L, the sodium bisulfite with the concentration of 1 mol/L, and the lipoprotein lipase with the concentration of 1 wt %.

In a second aspect, the disclosure provides an application method of the composition provided in the first aspect, including preserving the vitamin D and the vitamin K using the composition.

In an embodiment, the vitamin D includes at least one of vitamin D2 and vitamin D3. In an embodiment, the vitamin K includes vitamin K2.

In an embodiment, the vitamin K2 includes at least one of MK-4 and MK-7.

In an embodiment, the composition is used to preserve the vitamin D and the vitamin K in a sample to be tested, for example, to preserve the vitamin D and the vitamin K in serum after blood collection.

In an embodiment, the composition is used to preserve the vitamin D and the vitamin K in configured reagents, for example, to preserve the vitamin D and the vitamin K in reference standards used to construct standard curves during a detection process or quality control materials used for quality control.

In a third aspect, the disclosure provides a preservation method for the vitamin D and the vitamin K. The preservation method includes adding the composition provided in the first aspect to a system preserving the vitamin D and the vitamin K.

In an embodiment, the preservation method further includes reserving the vitamin D and the vitamin K under conditions of avoiding light and not exceeding 4 degrees Celsius (° C.).

In an embodiment, the vitamin D and the vitamin K are preserved at −20° C.

In an embodiment, the system preserving the vitamin D and the vitamin K is buffer, blood, plasma, or the serum, and more specifically, the system is the serum.

In a fourth aspect, the disclosure provides a surrogate matrix for the vitamin D and the vitamin K. The surrogate matrix includes the composition provided in the first aspect and a diluent.

In an embodiment, the diluent is the buffer, the blood, the plasma, or the serum, and more specifically, the diluent is the serum.

In an embodiment, the diluent is the blood, the plasma, or the serum from which the vitamin D and the vitamin K have been removed. The “from which the vitamin D and the vitamin K have been removed” refers to reducing contents of the vitamin D and the vitamin K inherent in the diluent, so as to reduce influence of the vitamin D and vitamin K inherent in the diluent on quantification of the vitamin D and vitamin K in the configured reagents. It should be understood that acceptable amounts of the vitamin D and the vitamin K are allowed to remain.

In an embodiment, the diluent is the serum from which the vitamin D and the vitamin K have been removed.

In an embodiment, the vitamin D and the vitamin K inherent in the serum are removed from the serum by solid-phase extraction, adsorbent adsorption, and ultraviolet irradiation.

In an embodiment, the serum sequentially undergoes the solid-phase extraction, the adsorbent adsorption, and the ultraviolet irradiation to remove the vitamin D and the vitamin K inherent in the serum.

In an embodiment, the solid-phase extraction is performed through a Strata® C8 solid-phase extraction column.

In an embodiment, an adsorbent for the adsorbent adsorption includes activated carbon coated with dextran.

In a fifth aspect, the disclosure provides a preparation method for the surrogate matrix for the vitamin D and the vitamin K. The preparation method includes mixing the composition provided in the first aspect with the diluent.

In an embodiment, the diluent is the serum from which the vitamin D and the vitamin K have been removed. The vitamin D and the vitamin K inherent in the serum are removed from the serum by the solid-phase extraction, the adsorbent adsorption, and the ultraviolet irradiation.

In an embodiment, the serum sequentially undergoes the solid-phase extraction, the adsorbent adsorption, and the ultraviolet irradiation to remove the vitamin D and the vitamin K inherent in the serum.

In an embodiment, the solid-phase extraction is performed through the Strata® C8 solid-phase extraction column.

In an embodiment, the adsorbent for the adsorbent adsorption includes the activated carbon coated with the dextran.

In a sixth aspect, the disclosure provides a reagent containing the vitamin D and the vitamin K. The reagent containing the vitamin D and the vitamin K includes predetermined amounts of the vitamin D and the vitamin K, and the surrogate matrix for the vitamin D and the vitamin K provided in the fourth aspect. The reagent includes but is not limited to the reference standards with different concentrations of the vitamin D and the vitamin K and the quality control materials with different concentrations of the vitamin D and the vitamin K.

In a seventh aspect, the disclosure provides a detection kit for the vitamin D and the vitamin K. The detection kit includes at least one of the composition provided in the first aspect, the surrogate matrix provided in the fourth aspect, and the reagent containing the vitamin D and the vitamin K provided in the sixth aspect.

In an eighth aspect, the disclosure provides a detection method for the vitamin D and the vitamin K. The detection method includes using the composition provided in the first aspect during the detection process to reduce loss of the vitamin D and the vitamin K in the sample to be tested. The detection method is a detection method for non-diagnostic and non-therapeutic purposes.

The loss of the vitamin D and the vitamin K includes degradation of the vitamin D and the vitamin K, and/or, the loss includes binding of the vitamin D and the vitamin K to substances in the environment such that the vitamin D and the vitamin K cannot be detected.

In an embodiment, the detection method includes using the reagent containing the vitamin D and the vitamin K provided in the sixth aspect to construct a standard curve.

In an embodiment, the detection method includes using chromatography to separate the vitamin D and the vitamin K from the sample to be tested.

In an embodiment, conditions of the chromatography include:
chromatography column: ACQUITY® UPLC® bridged ethylene hybrid (BEH) phenyl column (2.1 millimeters (mm)×50 mm, 1.7 micrometers (μm)); and
mobile phases: mobile phase A is a methanol solution containing ammonium fluoride (NH₄F) with a concentration of 0.5 millimoles per liter (mM) and formic acid with a concentration of 0.1% volume per volume (v/v); and mobile phase B is an aqueous solution containing formic acid with a concentration of 0.1% v/v.

An elution gradient is specifically as follows:
within 0 minutes (min) to 0.5 min, a volume ratio of the mobile phase A to the mobile phase B is maintained at 30:70;
within 0.5 min to 2 min, the volume ratio of the mobile phase A to the mobile phase B changes from 30:70 to 5:95;
within 2 min to 4.5 min, the volume ratio of the mobile phase A to the mobile phase B is maintained at 5:95;
within 4.5 min to 4.6 min, the volume ratio of the mobile phase A to the mobile phase B changes from 5:95 to 30:70; and
within 4.6 min to 5 min, the volume ratio of the mobile phase A to the mobile phase B is maintained at 30:70.

In an embodiment, the vitamin D and the vitamin K are detected by mass spectrometry.

In an embodiment, conditions of the mass spectrometry include: an ionization mode of electrospray ionization (positive mode), abbreviated as ESI (+); a detection method of multiple reaction monitoring (MRM); an ion source temperature (TEM) of 500° C.; a pressure of an atomized gas (Gas1) of 25.0 pounds per square inch (psi); a voltage (nebulizer current, abbreviated as NC) of 5.0 volts (V); and a pressure of a curtain gas of 25.0 psi.

The disclosure will be further described with reference to the embodiments; however, it should be understood that these embodiments are only used for more detailed description and should not be understood as limiting the disclosure in any way.

### Embodiment 1

Embodiment 1 provides a detection method for the vitamin D and the vitamin K.

### 1. Pretreatment Process of the Vitamin D and the Vitamin K

200 microliters (μL) of the serum, 20 μL of internal standard (vitamin D2-deuterium 6, vitamin D3-deuterium 5, vitamin MK-4-deuterium 7, and vitamin MK-7-deuterium 7), and 800 μL of ethanol-acetonitrile (1:1) are vortexed for 10 min and then centrifuged at a rotating speed of 14000 revolutions per minute (rpm) for 10 min to obtain a first supernatant. 700 μL of the first supernatant is mixed with 1300 μL hexane-isooctane (1:1, v/v) for extraction for 10 min and then centrifuged at 14000 rpm for 5 min to obtain a second supernatant. 1000 μL of the second supernatant is evaporated under nitrogen to obtain a concentrated mixture. The concentrated mixture is then resuspended in 80 μL of 80% ethanol and shaken for 2 min.

### 2. The Conditions of the Chromatography

The chromatography column is an ACQUITY® UPLC® BEH phenyl column (2.1 mm×50 mm, 1.7 μm). An injection volume is 5 μL. Mobile phases include the mobile phase A and the mobile phase B. The mobile phase A is the methanol solution containing the NH₄F with the concentration of 0.5 mM and the formic acid with the concentration of 0.1% v/v. The mobile phase B is the aqueous solution containing the formic acid with the concentration of 0.1% v/v. A flow velocity is 0.4 milliliters per minute (mL/min). A gradient elution procedure is shown in Table 1.

TABLE 1
Elution gradient

| Time (min) | Mobile phase A (% v/v) | Mobile phase B (% v/v) |
|---|---|---|
| 0 | 30 | 70 |
| 0.5 | 30 | 70 |
| 2 | 5 | 95 |
| 4.5 | 5 | 95 |
| 4.6 | 30 | 70 |
| 5 | 30 | 70 |

### 3. The Conditions of the Mass Spectrometry

The ionization mode is ESI (+). The detection method is MRM. The ion source temperature is 500° C. The pressure of the atomized gas (Gas1) is 25.0 psi. The voltage (NC) is 5.0 V. The pressure of the curtain gas is 25.0 psi. Corresponding MRM channels and parameters are shown in Table 2.

TABLE 2
Mass spectrometry parameters of components
to be tested and internal standards

| | | Dwell time | | | |
|---|---|---|---|---|---|
| First | Third | (second, | | Declustering | Collision |
| quadru- | quadru- | abbreviated | | potential | Energy |
| pole Q1 | pole Q3 | as s) | ID | (DP) | (CE) |
| 395.4 | 269.0 | 5 | VD2-1 | 85 | 14 |
| 395.4 | 211.0 | 5 | VD2-2 | 85 | 15 |
| 383.4 | 257.2 | 5 | VD3-1 | 70 | 15 |
| 383.4 | 365.4 | 5 | VD3-2 | 70 | 20 |
| 401.2 | 269.0 | 5 | VD2-IS | 85 | 14 |
| 388.1 | 262.3 | 5 | VD3-IS | 70 | 15 |
| 452.4 | 194.1 | 5 | MK-4-IS | 50 | 47 |
| 656.5 | 194 | 5 | MK-7-IS | 90 | 40 |
| 445.3 | 81.3 | 5 | MK-4-1 | 50 | 70 |
| 445.3 | 187.1 | 5 | MK-4-2 | 50 | 33 |
| 649.3 | 187.1 | 5 | MK-7-1 | 80 | 45 |
| 649.3 | 109.1 | 5 | MK-7-2 | 80 | 55 |

The following embodiments 2 to 4 all use the method of embodiment 1 for detecting vitamins in samples.

### Embodiment 2

This embodiment investigates methods for removing the vitamin D and the vitamin K from serum matrix, including using ion exchangers to remove trace elements from the serum to reduce influence of the trace elements on the vitamins, using ultrafiltration tubes to remove high molecular weight enzyme proteins to reduce influence of enzymes on the vitamins, using adsorbents to remove the vitamins from the serum, treating the serum with ultraviolet irradiation to degrade the vitamins, and using solid-phase extraction to remove the vitamins from the serum. Experimental groups are shown in Table 3.

TABLE 3
Experimental groups of removing the vitamin
D and the vitamin K from the serum matrix

| Experimental groups | Pretreatment method |
|---|---|
| Experimental group 1 | ion exchanger |
| Experimental group 2 | sequential ultrafiltration tube + ion exchanger |
| Experimental group 3 | sequential adsorbent + ion exchanger |
| Experimental group 4 | sequential ultraviolet irradiation + ion exchanger |
| Experimental group 5 | solid-phase extraction-Strata ® C8 |
| Experimental group 6 | solid-phase extraction-Strata ® Silica |
| Experimental group 7 | sequential solid-phase extraction-Strata ® C8 + |
| | adsorbent + ultraviolet irradiation |

Steps of each pretreatment method are as follows.

(1) Using the ion exchangers to remove the trace elements from the serum:
a weak cation exchange column, specifically an Oasis® WCX μElution Plate with a size of 30 μm;
activation: 200 μL of an aqueous solution containing 85% acetonitrile (ACN) and 1% formic acid (Fa);
equilibration: 200 μL of water;
loading: 600 μL of sample;
rinsing 1:200 μL of water;
rinsing 2:200 μL of ACN;
elution: 60 μL of the aqueous solution containing 85% ACN and 1% Fa; and
resuspension: 50 μL of a 0.01% vitamin C aqueous solution.

(2) Using the ultrafiltration tubes to remove the high molecular weight enzyme proteins Serum samples are treated using an Amicon® Ultra-15 centrifugal filter with a 50 kilo Dalton (kDa) molecular weight cutoff (Merck Millipore, cat. #UFC905096) for 30 min.

(3) Using the adsorbents to remove the vitamins from the serum

4 milliliters (mL) of ultrapure water, 0.1 grams (g) of activated carbon, and 0.01 g of dextran are added into a centrifuge tube and then shaken and centrifuged at a rotating speed of 18000 rpm for 5 min to obtain a first centrifuged product containing a third supernatant and activated carbon coated with the dextran. The third supernatant is discarded from the first centrifuged product to leave the activated carbon coated with the dextran. 5 mL of the serum is taken and added to drained activated carbon coated with the dextran, and then shaken and centrifuged at a rotating speed of 18000 rpm for 5 min to obtain a fourth supernatant. The fourth supernatant is taken and added to the drained activated carbon coated with the dextran, and then shaken and centrifuged at a rotating speed of 18000 rpm for 5 min to obtain a fifth supernatant. The fifth supernatant is collected to obtain the serum from which the vitamins are removed.

(4) Treating the serum with the ultraviolet irradiation to degrade the vitamins

Serum samples are irradiated under an ultraviolet lamp for 20 min to obtain the serum from which the vitamins are removed.

(5) Using the solid-phase extraction (Strata® C8 solid-phase extraction column) to remove the vitamins from the serum:
solid-phase extraction column: Strata® C8, 25 milligrams (mg), 8E-S005-CGB;
activation: methanol;
equilibration: water; and
loading: after centrifuging the serum to obtain a sixth supernatant, the sixth supernatant is taken and loaded onto a first solid-phase extraction (SPE) plate to receive a loading solution.

(6) Using the solid-phase extraction (Strata® Silica solid-phase extraction column) to remove the vitamins from the serum:
solid-phase extraction column: Strata® Silica, 50 mg, 8E-S012-DEG;
activation: normal hexane; and
loading: after centrifuging the serum to obtain a seventh supernatant, the seventh supernatant is taken and loaded onto a second SPE plate to receive the loading solution.

According to the experimental groups in Table 3, the vitamin D and the vitamin K are removed from the serum matrix added with reference standards. Recovery rates of vitamin D2, vitamin D3, vitamin K (MK-4), and vitamin K (MK-7) in treated serum matrix are detected, and detection results are shown in Table 4.

TABLE 4
Contents of the vitamin D and the vitamin K in the treated serum matrix

| | Recovery rate | Recovery rate | Recovery rate | Recovery rate |
|---|---|---|---|---|
| | of vitamin D2 | of vitamin D3 | of vitamin K | of vitamin K |
| Pretreatment method | (%) | (%) | (MK-4) (%) | (MK-7) (%) |
| Experimental group 1 | 76.3 | 81.2 | 45.3 | 39.6 |
| Experimental group 2 | 79.6 | 74.7 | 46.8 | 40.5 |
| Experimental group 3 | 67.4 | 72.3 | 24.6 | 15.7 |
| Experimental group 4 | 63.4 | 66.1 | 39.3 | 23.9 |
| Experimental group 5 | 16.2 | 14.7 | 6.1 | 2.4 |
| Experimental group 6 | 33.5 | 24.6 | 25.3 | 28.3 |
| Experimental group 7 | 4.2 | 6.4 | 0.7 | 1.1 |

It can be seen from the detection results shown in Table 4 that the ion exchangers and the ultrafiltration tubes cannot significantly remove the vitamin D and the vitamin K from the serum. The solid-phase extraction using the Strata® C8 solid-phase extraction column based on reverse-phase retention mode has a significant removal effect, and the best removal effect is achieved by combining the solid-phase extraction using the Strata® C8 solid-phase extraction column with the adsorbent and the ultraviolet irradiation. A combination of the solid-phase extraction-Strata® C8+adsorbent+ultraviolet irradiation can remove the vitamin D and the vitamin K from the serum.

### Embodiment 3

The embodiment investigates components of the composition for preserving the vitamin D and the vitamin K. The components to be investigated include oxidants, reducing agents, anti-adsorption agents, hydrolytic enzymes, and antibacterial agents, specifically including ProClin™ 300, the sodium metabisulfite, the sodium bisulfite, sodium azide, mannitol, and the lipoprotein lipase.

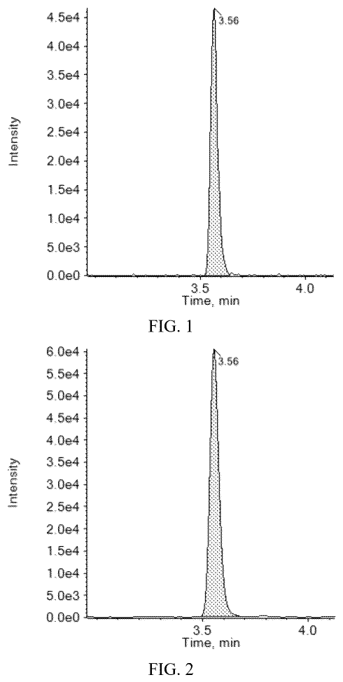

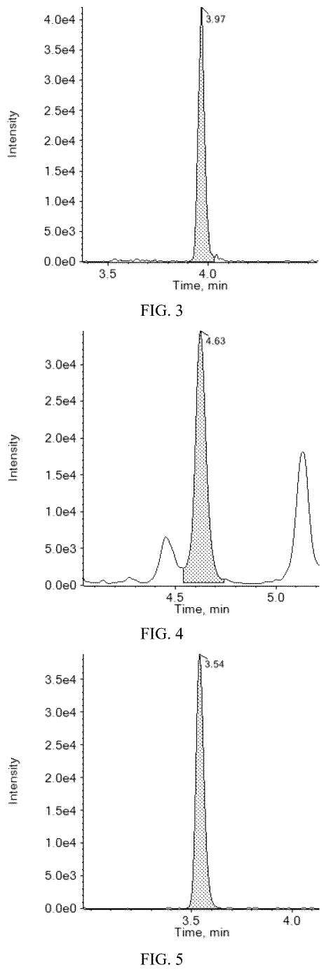

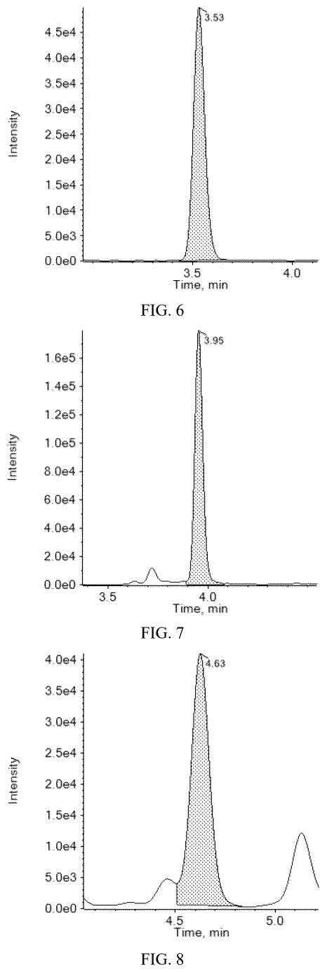

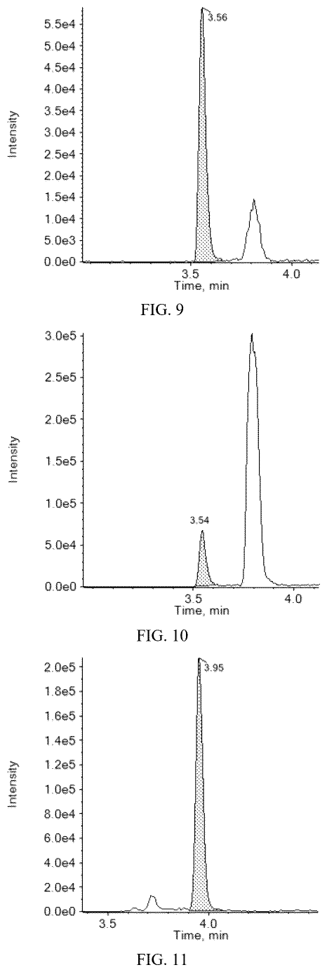

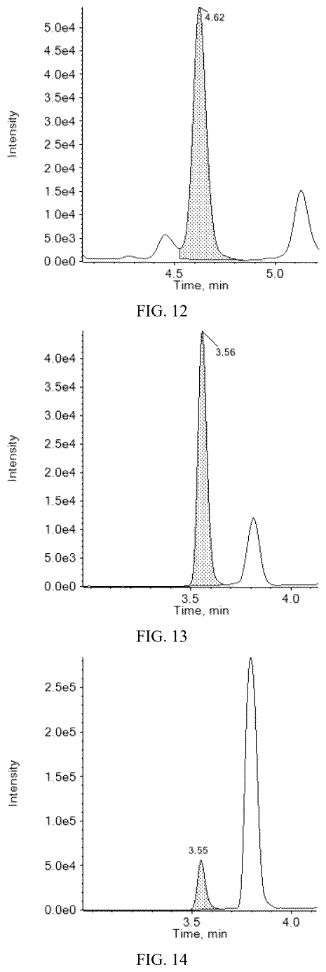

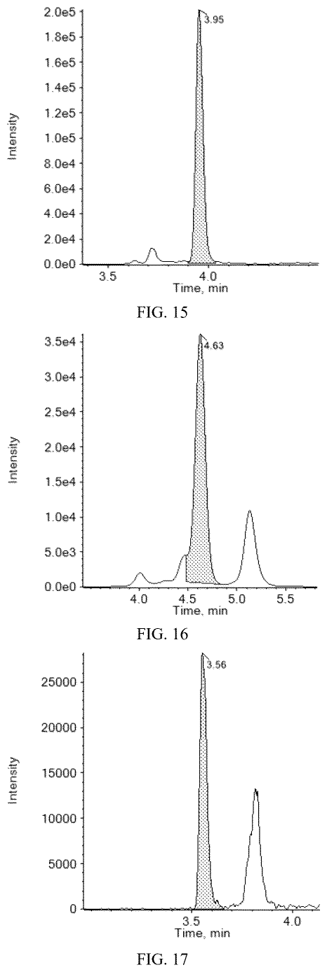

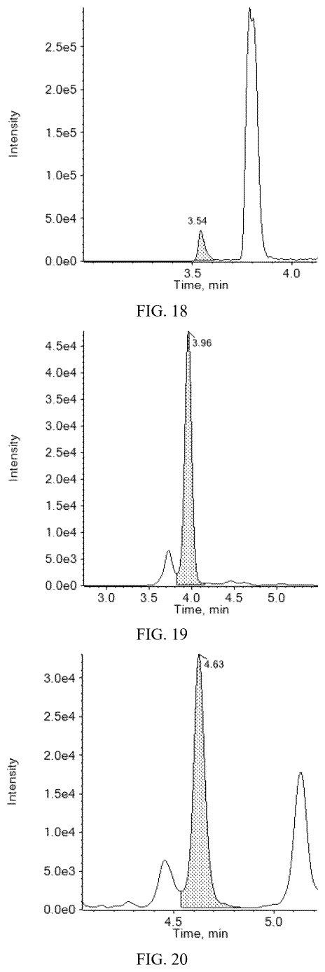

Combinations prepared using the above six substances are added to serum samples. Reference standards are added to the serum samples added with the combinations. The serum samples added with the combinations and the reference standards are placed under light at room temperature for one day to measure the recovery rates of the vitamin D and the vitamin K. Measurement results of the recovery rates are shown in Table 6. Chromatograms of the vitamin D2, the vitamin D3, the vitamin K (MK-4), and the vitamin K (MK-7) in experimental groups 3, 7, 8, 12, and 16 are illustrated in FIG. 1 through FIG. 20.

TABLE 5
Components of combinations

| | Components of combinations and final concentration of each of |
|---|---|
| Experimental groups | the components |
| Experimental group 1 | None of the components to be investigated is added. |
| Experimental group 2 | 0.1% ProClin ™ 300 |
| Experimental group 3 | 1 mole per liter (M) sodium metabisulfite |
| Experimental group 4 | 1M sodium bisulfite |
| Experimental group 5 | 1M sodium azide |
| Experimental group 6 | 0.2 wt % mannitol |
| Experimental group 7 | 1 wt % lipoprotein lipase |
| Experimental group 8 | 1M sodium metabisulfite + 1M sodium bisulfite + 1 wt % |
| | lipoprotein lipase |
| Experimental group 9 | 0.5M sodium metabisulfite + 2M sodium bisulfite + 0.5 wt % |
| | lipoprotein lipase |
| Experimental group 10 | 2M sodium metabisulfite + 0.5M sodium bisulfite + 2 wt % |
| | lipoprotein lipase |
| Experimental group 11 | 0.5M sodium metabisulfite + 3M sodium bisulfite + 0.5 wt % |
| | lipoprotein lipase |
| Experimental group 12 | 3M sodium metabisulfite + 0.5M sodium bisulfite + 3 wt % |
| | lipoprotein lipase |
| Experimental group 13 | 1M sodium metabisulfite + 1M sodium bisulfite |
| Experimental group 14 | 1M sodium bisulfite + 1 wt % lipoprotein lipase |
| Experimental group 15 | 1M sodium metabisulfite + 1 wt % lipoprotein lipase |
| Experimental group 16 | 1M sodium metabisulfite + 1M sodium bisulfite + 0.2 wt % |
| | mannitol |
| Experimental group 17 | 1M sodium metabisulfite + 1M sodium bisulfite + 1M sodium |
| | azide |

TABLE 6
Recovery rate of each vitamin after adding
the combinations of the experimental groups

| | Recovery rate (%) | | | | | |
|---|---|---|---|---|---|---|
| Experimental groups | VD2 | VD3 | MK-4 | MK-7 | VA | VB1 |
| Experimental group 1 | 78.4 | 67.3 | 60.6 | 49.3 | 47.3 | 52.7 |
| Experimental group 2 | 84.2 | 85.3 | 63.7 | 58.5 | 43.8 | 49.6 |
| Experimental group 3 | 88.4 | 90.7 | 58.3 | 64.5 | 54.3 | 56.4 |
| Experimental group 4 | 93.5 | 83.1 | 76.4 | 67.4 | 48.9 | 53.0 |
| Experimental group 5 | 67.3 | 64.5 | 53.6 | 55.2 | 45.2 | 54.2 |
| Experimental group 6 | 43.6 | 55.7 | 58.4 | 44.7 | 46.8 | 55.3 |
| Experimental group 7 | 78.3 | 80.3 | 81.5 | 74.6 | 41.6 | 50.5 |
| Experimental group 8 | 95.8 | 93.6 | 86.4 | 88.6 | 57.6 | 59.3 |
| Experimental group 9 | 90.5 | 91.3 | 81.9 | 82.1 | 52.7 | 55.3 |
| Experimental group 10 | 91.1 | 90.4 | 81.6 | 80.4 | 55.3 | 50.4 |
| Experimental group 11 | 83.6 | 84.2 | 72.4 | 78.3 | 47.6 | 52.6 |
| Experimental group 12 | 84.6 | 86.3 | 73.2 | 71.5 | 50.1 | 57.3 |
| Experimental group 13 | 82.4 | 85.3 | 73.2 | 70.4 | 46.5 | 49.2 |
| Experimental group 14 | 79.3 | 77.5 | 77.2 | 74.2 | 47.2 | 53.7 |
| Experimental group 15 | 80.3 | 74.5 | 72.4 | 70.5 | 47.5 | 48.7 |
| Experimental group 16 | 63.2 | 59.3 | 60.4 | 58.3 | 51.3 | 53.9 |
| Experimental group 17 | 61.5 | 60.2 | 63.6 | 65.4 | 50.6 | 55.2 |

It can be seen from the above experiment results that, a combination of the sodium metabisulfite, the sodium bisulfite, and the lipoprotein lipase has the best protective effect on the vitamin D and the vitamin K, superior to using only two or one of these three components in combination. Moreover, replacing the lipoprotein lipase with the mannitol or the sodium azide has a lower protective effect on the vitamin D and the vitamin K than the combination of the sodium metabisulfite, the sodium bisulfite, and the lipoprotein lipase. Among them, optimal concentrations are 1 M for the sodium metabisulfite, 1 M for the sodium bisulfite, and 1 wt % for the lipoprotein lipase. Furthermore, it can be seen from experimental results shown in Table 4 that the combination of the sodium metabisulfite, the sodium bisulfite, and the lipoprotein lipase is more suitable for alleviating degradation of the vitamin D and the vitamin K, but exhibits no significant advantage in protective effect on vitamin A and vitamin B1 compared to other combinations.

### Embodiment 4

This embodiment investigates effects of light exposure and storage temperature on the degradation of the vitamin D and the vitamin K in the serum. An experimental method is as follows.

1. Low-value quality control materials (QCL), medium-value quality control materials (QCM), and high-value quality control materials (QCH) are configured. The vitamin D, the vitamin D3, the vitamin K (MK-4), and the vitamin K (MK-7) are added to these quality control materials to reach target concentrations. These quality control materials added with the vitamins are grouped according to the following conditions: experimental group 1: light-shielded freezing (−20° C.); experimental group 2: light-shielded refrigeration (−4° C.); experimental group 3: light-shielded room temperature (25° C.); experimental group 4: room temperature under light exposure (25° C.). These quality control materials added with the vitamins in different experimental groups are stored for one day, two days, and seven days, respectively, to measure the recovery rates of the vitamin D, the vitamin D3, the vitamin K (MK-4), and the vitamin K (MK-7). Measurement results of the recovery rates are shown in Table 7 through Table 10.

TABLE 7
Experimental results of experimental group 1

| | |
|---|---|
| Storage | Recovery rate (%) |

| time | Analyte | MK-4 | MK-7 | VD2 | VD3 |
|---|---|---|---|---|---|
| One | QCL | 94.1 | 87.59 | 103.74 | 113.17 |
| day | QCM | 110.94 | 99.11 | 89.98 | 92.5 |
| | QCH | 106.99 | 86.05 | 111.17 | 100.66 |
| Two | QCL | 106.15 | 91.67 | 107 | 69.6 |
| days | QCM | 125.5 | 87.89 | 98.84 | 103.41 |
| | QCH | 104.52 | 85.26 | 69.97 | 122.01 |
| Seven | QCL | 90.35 | 92.26 | 100.17 | 101.2 |
| days | QCM | 85.2 | 85.47 | 102.45 | 113.22 |
| | QCH | 93.93 | 88.01 | 96.09 | 112.42 |

TABLE 8
Experimental results of experimental group 2

| | |
|---|---|
| Storage | Recovery rate (%) |

| time | Analyte | MK-4 | MK-7 | VD2 | VD3 |
|---|---|---|---|---|---|
| One | QCL | 92.3 | 99.1 | 96.6 | 95.6 |
| day | QCM | 97.5 | 95.2 | 103.4 | 99.7 |
| | QCH | 103.6 | 107.3 | 104.9 | 100.9 |
| Two | QCL | 99.7 | 99.9 | 100.6 | 102.7 |
| days | QCM | 105 | 101.2 | 96.05 | 98.21 |
| | QCH | 91.83 | 91.06 | 93.25 | 96.39 |
| Seven | QCL | 71.8 | 62.03 | 79.49 | 88.12 |
| days | QCM | 43.93 | 61.44 | 80.44 | 71.43 |
| | QCH | 74.29 | 58.44 | 77.24 | 65.83 |

TABLE 9
Experimental results of experimental group 3

| | |
|---|---|
| Storage | Recovery rate (%) |

| time | Analyte | MK-4 | MK-7 | VD2 | VD3 |
|---|---|---|---|---|---|
| One | QCL | 63.7 | 88.55 | 94.28 | 85.4 |
| day | QCM | 78.69 | 89.96 | 110.34 | 88.86 |
| | QCH | 83.42 | 78.84 | 110.75 | 109.45 |
| Two | QCL | 43.04 | 70.73 | 85.95 | 80.06 |
| days | QCM | 56.73 | 71.43 | 85.1 | 86.51 |
| | QCH | 46.32 | 76.07 | 80.97 | 79.47 |
| Seven | QCL | 22.51 | 31.64 | 64.73 | 61.58 |
| days | QCM | 23.34 | 23.22 | 55.69 | 69.94 |
| | QCH | 23.67 | 29.04 | 61.79 | 61.44 |

TABLE 10
Experimental results of experimental group 4

| | |
|---|---|
| Storage | Recovery rate (%) |

| time | Analyte | MK-4 | MK-7 | VD2 | VD3 |
|---|---|---|---|---|---|
| One | QCL | 61.62 | 64.49 | 87.23 | 90.3 |
| day | QCM | 72.21 | 62.22 | 106.83 | 102.83 |
| | QCH | 78.23 | 65.93 | 108.43 | 115.66 |
| Two | QCL | 39.65 | 40.78 | 80.6 | 79.63 |
| days | QCM | 37.92 | 44.48 | 79.42 | 86.53 |
| | QCH | 36.09 | 46.29 | 84.48 | 79.84 |
| Seven | QCL | 19.38 | 28.51 | 69.84 | 58.73 |
| days | QCM | 13.17 | 24.43 | 63.86 | 60.88 |
| | QCH | 18.32 | 22.01 | 63.11 | 69.57 |

It can be seen from Table 7 through Table 10 that storage temperature significantly affects stability of the vitamin K (MK-4), and light significantly affects stability of vitamin K (MK-7). Therefore, the serum samples need to be stored away from light and frozen after blood collection.

Finally, it should be noted that the above embodiments are only used to illustrate the technical solution of the disclosure, and not to limit the disclosure. Although the disclosure has been described in detail with reference to the aforementioned embodiments, those skilled in the art should understand that they can still make modifications to the technical solutions described in the aforementioned embodiments, or make equivalent substitutions to some or all of the technical features, and these modifications or substitutions do not deviate essence of corresponding technical solutions from a scope of the various embodiments of the disclosure.

What is claimed is:

1. A composition for preserving vitamin D and vitamin K, comprising sodium metabisulfite with a concentration in a range of 0.5 moles per liter (mol/L) to 3 mol/L, sodium bisulfite with a concentration in a range of 0.5 mol/L to 3 mol/L, and lipoprotein lipase with a concentration in a range of 0.5 weight percent (wt %) to 3 wt %.

2. The composition as claimed in claim 1, comprising the sodium metabisulfite with the concentration in a range of 0.5 mol/L to 2 mol/L, the sodium bisulfite with the concentration in a range of 0.5 mol/L to 2 mol/L, and the lipoprotein lipase with the concentration in a range of 0.5 wt % to 2 wt %.

3. The composition as claimed in claim 2, comprising the sodium metabisulfite with the concentration of 1 mol/L, the sodium bisulfite with the concentration of 1 mol/L, and the lipoprotein lipase with the concentration of 1 wt %.

4. An application method of the composition as claimed in claim 1, comprising:
preserving the vitamin D and the vitamin K using the composition;
wherein the vitamin D comprises at least one of vitamin D2 and vitamin D3;
wherein the vitamin K comprises vitamin K2; and
wherein the vitamin K2 comprises at least one of menaquinone-4 and menaquinone-7.

5. A preservation method for the vitamin D and the vitamin K, comprising adding the composition as claimed in claim 1 to a system preserving the vitamin D and the vitamin K;
wherein the preservation method further comprises preserving the vitamin D and the vitamin K under conditions of avoiding light and not exceeding 4 degrees Celsius (° C.); and
wherein the system preserving the vitamin D and the vitamin K is buffer, blood, plasma, or serum.

6. A surrogate matrix for the vitamin D and the vitamin K, comprising the composition as claimed in claim 1 and a diluent;
wherein the diluent is buffer, blood, plasma, or serum.

7. The surrogate matrix as claimed in claim 6, wherein the diluent is the blood, the plasma, or the serum from which the vitamin D and the vitamin K have been removed.

8. A preparation method for the surrogate matrix for the vitamin D and the vitamin K as claimed in claim 6, comprising mixing the composition with the diluent;
wherein the diluent is the serum from which the vitamin D and the vitamin K have been removed; and the vitamin D and the vitamin K are removed from the serum by at least one method selected from the group consisting of solid-phase extraction, adsorbent adsorption, and ultraviolet irradiation; and
wherein an adsorbent for the adsorbent adsorption comprises activated carbon coated with dextran.

9. A reagent containing the vitamin D and the vitamin K, comprising predetermined amounts of the vitamin D and the vitamin K, and the surrogate matrix for the vitamin D and the vitamin K as claimed in claim 6.

10. A detection kit for the vitamin D and the vitamin K, comprising the composition as claimed in claim 1.

11. A detection kit for the vitamin D and the vitamin K, comprising the surrogate matrix for the vitamin D and the vitamin K as claimed in claim 6.

12. A detection kit for the vitamin D and the vitamin K, comprising the reagent as claimed in claim 9.

13. A detection method for the vitamin D and the vitamin K, comprising using the composition during a detection process to reduce loss of the vitamin D and the vitamin K in a sample; and
wherein a standard curve is constructed using the reagent as claimed in claim 9.

14. The detection method as claimed in claim 13, wherein the detection method comprises using liquid chromatography to separate the vitamin D and the vitamin K from the sample to be tested.
